(19) United States
(12) Patent Application Publication
(10) Pub. No.: US 2021/0153747 A1
(43) Pub. Date: May 27, 2021

(54) MOBILE DEVICE FOR DETECTING ORAL PATHOLOGY

(71) Applicant: METAL INDUSTRIES RESEARCH & DEVELOPMENT CENTRE, Kaohsiung (TW)

(72) Inventors: Keng-Ta LIN, Kaohsiung City (TW); Po-Chi HU, KAOHSIUNG CITY (TW); Yuan-Hsun TSAI, Kaohsiung City (TW); Zong-Hsin LIU, Kaohsiung City (TW)

(21) Appl. No.: 17/037,664

(22) Filed: Sep. 29, 2020

(30) Foreign Application Priority Data
Nov. 27, 2019 (TW) 108143251

(51) Int. Cl. A61B 5/00

(52) U.S. Cl. CPC A61B 5/0088; A61B 5/0086; A61B 5/7425; A61B 5/7445; A61B 5/0077

(57) ABSTRACT

A mobile device for detecting an oral pathology includes a casing, a probing unit, and a processor. The probing unit includes a fiber bundle set and a contact part configured to contact a gum portion of a tooth of an examinate. The fiber bundle set includes a light source fiber bundle and a light-receiving fiber bundle. The light source fiber bundle has a light-exiting end within the contact part, and can project a probing light onto the gum portion. The light-receiving fiber bundle has a light-receiving end within the contact part. The light-receiving fiber bundle can receive diffuse reflection lights generated after the probing light is diffuse reflected by the gum portion. The processor is in signal connection with the light-receiving fiber bundle, and can receive the diffuse reflection lights, to build an optical spectrum, and to determine a state of the gum portion according to the optical spectrum.

### RELATED APPLICATIONS

This application claims priority to Taiwan Application Serial Number 108143251, filed Nov. 27, 2019, which is herein incorporated by reference.

### BACKGROUND

### Field of Invention

The present invention relates to an oral pathology detection device. More particularly, the present invention relates to a mobile device for detecting an oral pathology.

### Description of Related Art

Oral diseases have affected most of the global population, and a serious periodontal disease is a very common disease. The periodontal disease is a periodontal tissue inflammation caused by a large number of germs reproducing and accumulating on periphery of a gum. When the periodontal disease is mild, an edge of the gum is inflamed, the gum is atrophied, and roots are exposed; and when the periodontal disease is exacerbated, the germs grow deep into the roots, such that the teeth are loose and shifted, and even loss due to losing support.

Currently, there is a prober for measuring periodontal pockets, in which a probing portion of the prober is flat. The flat prober design can disperse force to a gum while the prober goes deep into the packet to reduce pain of a patient. However, in measuring, the prober needs to be inserted between periodontal pockets of teeth and a gum, and needs to go deep into the periodontal pocket of the teeth to measure one after another, such that unbearable pain is still caused after measuring for a period of time, and thus fearing the patient.

There is another handheld device for detecting dental plaque, which includes an optical filter, and the optical filter can filter out blue light in longwave ultraviolet light emitted by light-emitting diodes to enhance recognition of the dental plaque with human eyes, so as to increase detection quality. It is convenient for the general public to hold the handheld device with one hand to do dental plaque detection on teeth by himself, such that it becomes easier for human to recognize dental plaque. However, the handheld device only can check the surfaces of the teeth, and cannot detect the gums, such that estimation and judgement of periodontal diseases cannot be done exactly. In addition, the handheld device does not have an image recording function, which is disadvantageous to marking and counting of the dental plaque.

### SUMMARY

Therefore, one objective of the present invention is to provide a mobile device for detecting an oral pathology, in which a probing unit recognizes an inflamed tissue feature of a gum by using a diffuse reflection principle, such that it needs not to be inserted into a gum space while detecting a state of the gum, thereby significantly easing discomfort of an examinate.

Another objective of the present invention is to provide a mobile device for detecting an oral pathology, which may include a detection unit for inspecting dental plaque, such that the mobile device can firstly check if the teeth get a periodontal disease caused by the dental plaque, and then detect gingivitis. Therefore, the mobile device for detecting an oral pathology can do more comprehensive oral detection to an examinate.

Still another objective of the present invention is to provide a mobile device for detecting an oral pathology, in which a processor can be connected to a healthcare information system (HIS) and an information and communications technology (ICT) system of a hospital, and can fill the relative probe and detection data out periodontal digital medical record forms which are also connected to the healthcare information system and the information and communications technology system of the hospital. Therefore, time required for filling out the form in detection is greatly reduced, and it is beneficial to perfect a treatment solution of an examinate after detecting.

According to the aforementioned objectives, the present invention provides a mobile device for detecting an oral pathology. The mobile device for detecting an oral pathology includes a casing, a probing unit, and a processor. The probing unit is connected to the casing and includes at least one fiber bundle set and at least one contact part. Each contact part is configured to contact a gum portion of a tooth of an examinate. Each fiber bundle set includes a light source fiber bundle and a light-receiving fiber bundle. The light source fiber bundle has a light-exiting end, in which the light-exiting end is disposed within the contact part. The light source fiber bundle is configured to project a probing light onto the gum portion. The light-receiving fiber bundle is adjacent to the light source fiber bundle and has a light-receiving end, in which the light-receiving end is disposed within the contact part. The light-receiving fiber bundle is configured to receive diffuse reflection lights which are generated after the probing light is diffuse reflected by the gum portion. The processor is disposed within the casing and is in signal connection with the light-receiving fiber bundle. The processor is configured to receive the diffuse reflection lights, to build an optical spectrum by using the diffuse reflection lights, and to determine a state of the gum portion according to the optical spectrum.

According to one embodiment of the present invention, the at least one contact part is a sheath structure, and the at least one contact part is configured to cap the tooth of the examinate and cover the gum portion.

According to one embodiment of the present invention, a quantity of the at least one fiber bundle set is 6, and a quantity of the at least one contact part is 1, the fiber bundle sets are equally divided into two groups which are respectively disposed in two opposite sides of the contact part, and the two sides of the contact part respectively correspond to a buccal side and a lingua side of the tooth.

According to one embodiment of the present invention, the light source fiber bundle is an infrared light source fiber bundle, and the probing light is an infrared light.

According to one embodiment of the present invention, the processor stores a gingival diffuse reflection spectrum data base, and the processor matches the optical spectrum using the gingival diffuse reflection spectrum data base to determine the state of the gum portion.

According to one embodiment of the present invention, the processor is configured to connect to a healthcare information system and an information and communications technology system, and the processor is further configured to fill the state of the gum portion out a periodontal digital medical record form via the healthcare information system and the information and communications technology system.

According to one embodiment of the present invention, the mobile device for detecting an oral pathology further includes a detection unit. The detection unit includes an image-taking lens, an image-taking module, and a light source module. The image-taking lens is disposed on one side of the casing and is configured to get various oral images of the examinate. The image-taking module is disposed within the casing and is in signal connection with the image-taking lens to obtain the oral images. The light source module is adjacent to the image-taking lens and is configured to provide the image-taking lens with light.

According to one embodiment of the present invention, the light source module includes various first light-emitting diodes and various second light-emitting diodes, the first light-emitting diodes and the second light-emitting diodes alternate with each other and surround the image-taking lens, and the first light-emitting diodes and the second light-emitting diodes have different emission wavelengths.

According to one embodiment of the present invention, the first light-emitting diodes are various ultraviolet light-emitting diodes, and the second light-emitting diodes are various white light-emitting diodes.

According to one embodiment of the present invention, the mobile device for detecting an oral pathology further includes a display device, a handle, and a cover. The display device is disposed on another side of the casing and is in signal connection with the processor. The display device is configured to display the oral images and the state of the gum portion. The handle is connected to the casing. The cover is disposed around the image-taking lens and the light source module.

### BRIEF DESCRIPTION OF THE DRAWINGS

The invention can be more fully understood by reading the following detailed description of the embodiment, with reference made to the accompanying drawings as follows:

FIG. 1 is a three-dimensional schematic diagram of a mobile device for detecting an oral pathology in accordance with one embodiment of the present invention;

FIG. 2 is a schematic diagram of interior fittings of a mobile device for detecting an oral pathology after a casing is removed in accordance with one embodiment of the present invention;

FIG. 3 is a schematic diagram of a detection device of a mobile device for detecting an oral pathology in detection in accordance with one embodiment of the present invention; and

FIG. 4 is a schematic front view of an image-taking lens, a light source module, and a cover of a mobile device for detecting an oral pathology in accordance with one embodiment of the present invention.

### DETAILED DESCRIPTION

The terms “first”, “second”, and the like, as used herein, are not intended to mean a sequence or order, and are merely used to distinguish elements or operations described in the same technical terms.

In view of the exited oral detection device only have one single function of detecting dental plaque on surfaces of teeth or detecting gingivitis, a flat prober for measuring periodontal pockets can disperse force to a gum applied while the flat prober is inserted the packet but unbearable pain is still caused, and a statistical form needed to be filled out manually during detection which is disadvantageous to oral detection. Therefore, the present invention provides a mobile device for detecting an oral pathology herein, in which a probing unit needs not to be inserted into a gum space while detecting a gum state, thereby significantly easing discomfort of an examinate. In addition, the mobile device for detecting an oral pathology may include a detection unit for inspecting dental plaque, such that the mobile device for detecting an oral pathology can do more comprehensive oral detection to an examinate. Furthermore, a processor of the mobile device for detecting an oral pathology can be connected to a healthcare information system and an information and communications technology system of a hospital, and can fill the probe and detection data out periodontal digital medical record forms which are also connected to the healthcare information system and the information and communications technology system of the hospital. Therefore, time required for filling out the form in detection is greatly reduced, and it is beneficial to perfect a treatment solution of an examinate after detecting.

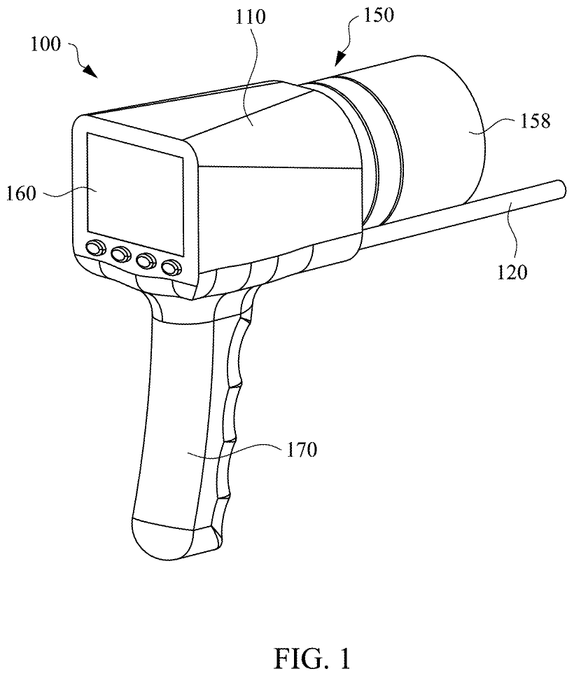

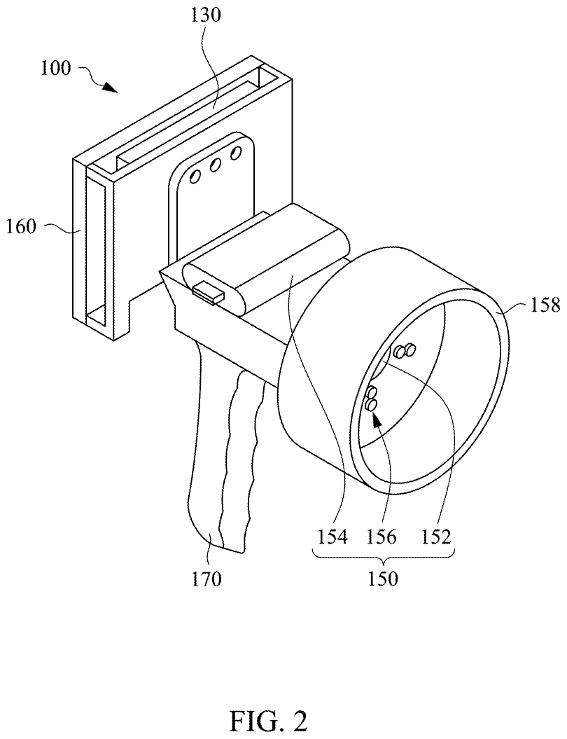

Referring to FIG. 1 and FIG. 2, which respectively illustrate a three-dimensional schematic diagram of a mobile device for detecting an oral pathology and a schematic diagram of interior fittings of the mobile device for detecting an oral pathology after a casing is removed in accordance with one embodiment of the present invention. A mobile device 100 for detecting an oral pathology mainly includes a casing 110, a probing unit 120, and a processor 130. The probing unit 120 is connected to the casing 110 and is protruded from one side of the casing 110. For example, as shown in FIG. 1, the probing unit 120 is protruded from a front side of the casing 110.

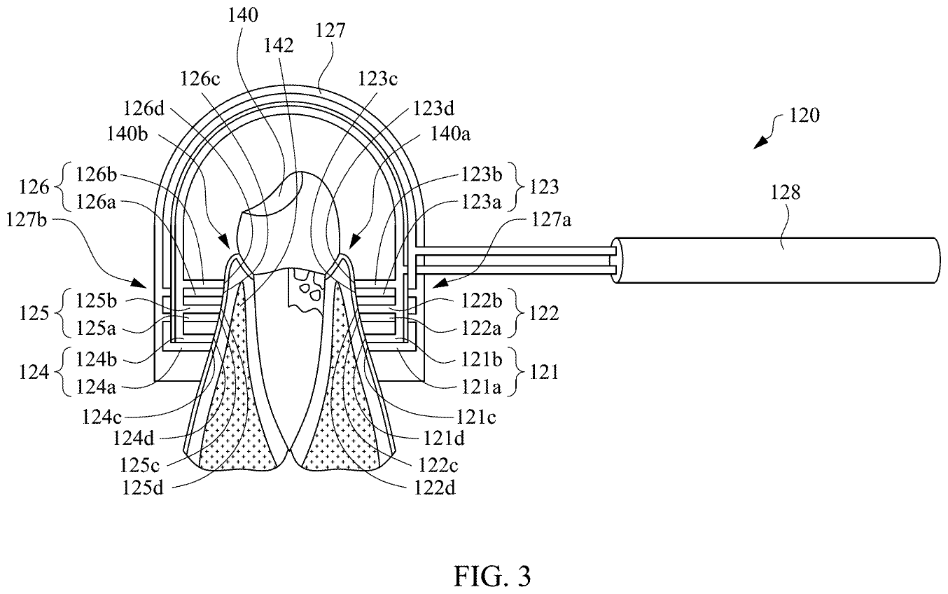

Referring to FIG. 3 simultaneously, FIG. 3 is a schematic diagram of a detection device of a mobile device for detecting an oral pathology in detection in accordance with one embodiment of the present invention. The probing unit 120 may be used to detect a state of a gum of a tooth of an examinate. For example, the probing unit 120 may be used to detect if the examinate gets gingivitis. The probing unit 120 mainly includes at least one fiber bundle set and at least one contact part. In some examples, as shown in FIG. 3, the probing unit 120 includes six fiber bundle sets 121-126 and one contact part 127. The contact part 127 is used to contact a gum portion 142 of a tooth 140 of an examinate. A surface of the contact part 127 may be silicone rubber, for example. In the examples, the contact part 127 is a sheath structure, and the contact part 127 may cap the tooth 140 of the examinate and cover the gum portion 140.

As shown in FIG. 3, middle sections and rear sections of the six fiber bundle sets 121-126 may be packed and integrated by using a flexible hose 128, and front sections of the fiber bundle sets 121-126 are separated from each other and are individually moved. The six fiber bundle sets 121-126 may be polygons, circles, or any combinations thereof. The six fiber bundle sets 121-126 may be equally divided into two groups, i.e. the fiber bundle sets 121-123 and fiber bundle sets 124-126, and the two groups of the fiber bundle sets 121-123 and the fiber bundle sets 124-126 are respectively penetrated into two opposite sides 127a and 127b of the contact part 127. When the tooth 140 is sheathed with the contact part 127, the two sides 127a and 127b are located at a buccal side 140a and a lingua side 140b of the tooth 140 correspondingly. In some exemplary examples, the fiber bundle sets 121-123 are respectively near a lower area, a middle area, and an upper area of the gum portion 142 at the buccal side 140a of the tooth 140, and the fiber bundle sets 124-126 are respectively near a lower area, a middle area, and an upper area of the gum portion 142 at the buccal side 140b of the tooth 140.

The fiber bundle set 121 includes a light source fiber bundle 121a and a light-receiving fiber bundle 121b. Similarly, the fiber bundle sets 122-126 respectively include light source fiber bundles 122a-126a and light-receiving fiber bundles 122b-126b. The light source fiber bundles 121a-126a respectively have light-exiting ends 121c-126c, in which the light-exiting ends 121c-126c are respectively on front ends of the light source fiber bundles 121a-126a. The light-exiting ends 121c-126c are disposed within the contact part 127. The light source fiber bundles 121a-126a project probing light onto the gum portion 142 through their light-exiting ends 121c-126c respectively. In some exemplary examples, the light source fiber bundles 121a-126a are infrared light source fiber bundles, and the probing light projected to the gum portion 142 by the light source fiber bundles 121a-126a is infrared light. After the probing light projected by the light source fiber bundles 121a-126a enters soft tissues like the gum portion 142, diffuse reflection lights are generated when the probing light are reflected by the different structures, such as the gum and the teeth. The infrared light has good transmittance to the soft tissues, and is beneficial to the probing of the gum portion 142.

In the fiber bundle sets 121-126, the light-receiving fiber bundles 121b-126b are respectively adjacent to the light source fiber bundles 121a-126a. The light-receiving fiber bundles 121b-126b respectively have light-receiving ends 121d-126d, in which the light-receiving ends 121d-126d are respectively located on front ends of the light-receiving fiber bundles 121b-126b. The light-receiving ends 121d-126d are similarly disposed within the contact par 127. The light-receiving fiber bundles 121b-126b receive diffuse reflection lights which are generated after the probing light projected by the light source fiber bundles 121a-126a is diffuse reflected by the gum portion 142 through their light-receiving ends 121d-126d. Thus, these fiber bundle sets 121-126 can form different lighting zones and light-receiving zones corresponding to the lighting zones. In some exemplary examples, each of the light-receiving fiber bundles 121b-126b may be separated from the corresponding light source fiber bundles 121a-126a with a predetermined distance, and each of the light-receiving fiber bundles 121b-126b and the corresponding light source fiber bundles 121a-126a may have a predetermined included angle.

In other examples, the probing unit may only include one fiber bundle set and one contact part. The fiber bundle set similarly includes a light source fiber bundle and a light-receiving fiber bundle, in which a light-exiting end of the light source fiber bundle and a light-receiving end of the light-receiving fiber bundle are adjacent to each other and disposed within the contact part. The fiber bundle set may be packed by using a flexible hose. By using a front end of the probing unit to contact any areas of a gum portion of a tooth of an examinate to adjoin the light-exiting end of the light source fiber bundle and the light-receiving end of the light-receiving fiber bundle to the contacted area of the gum portion, diffuse reflection lights of the area of the gum portion can be obtained.

In still other examples, the probing unit may include various fiber bundle sets and various contact parts, in which the fiber bundle sets and the contact parts has the same quantity. Light-exiting ends of light source fiber bundles the fiber bundle sets and light-receiving ends of light-receiving fiber bundles are respectively corresponding to and adjacent to each other and are correspondingly disposed within the contact parts. By using the contact parts of the probing unit to contact various areas of gum portions of teeth of an examinate, diffuse reflection lights of the areas of the gum portions can be obtained.

Referring to FIG. 1 and FIG. 2 simultaneously, the processor 130 is disposed within the casing 110 and is in signal connection with the light-receiving fiber bundles 121b-126b. For example, the processor 130 may be in signal connection with the light-receiving fiber bundles 121b-126b by electric wires or a wireless transmission method. The processor 130 can receive the diffuse reflection lights which are received by the light-receiving fiber bundles 121b-126b via signal transmission. The processor 130 may further build corresponding optical spectrums by using the diffuse reflection lights transmitted by the light-receiving fiber bundles 121b-126b respectively. The processor 130 may further determine states, such as inflammation states or healthy states, of the areas of the gum portion 142 probed by the fiber bundle sets 121-126 according to the optical spectrums. For example, the processor 130 may be a central processing unit (CPU) having an image processing function and a calculation and logical judgment function.

In some exemplary examples, the processor 130 may store a gingival diffuse reflection spectrum data base, in which the gingival diffuse reflection spectrum data base includes diffuse reflection spectrum data for lesion feature recognition. In such examples, the processor 130 can match the optical spectrum using the gingival diffuse reflection spectrum data base to determine the states of the various areas of the gum portion 142.

In some examples, the processor 130 may be connected to the healthcare information system and the information and communications technology system by a wired transmission technique or a wireless transmission technique. In these examples, the processor b130 may further fill the determined states of the various areas of the gum portion 142 out the periodontal digital medical record form via the healthcare information system and the information and communications technology system, in which the periodontal digital medical record form is simultaneously connected to the healthcare information system and the information and communications technology system. Accordingly, time required for filling out the form in detection is greatly reduced, and patients matching condition can enter a special periodontal care payment object treatment list whose fees are paid by Government, thereby perfecting a treatment solution after detecting.

The probing unit 120 needs not to be inserted into a gum space of an examinate while detecting a gum state of teeth of the examinate, such that discomfort of the examinate is significantly eased to increase check willingness, thereby effectively reducing oral diseases.

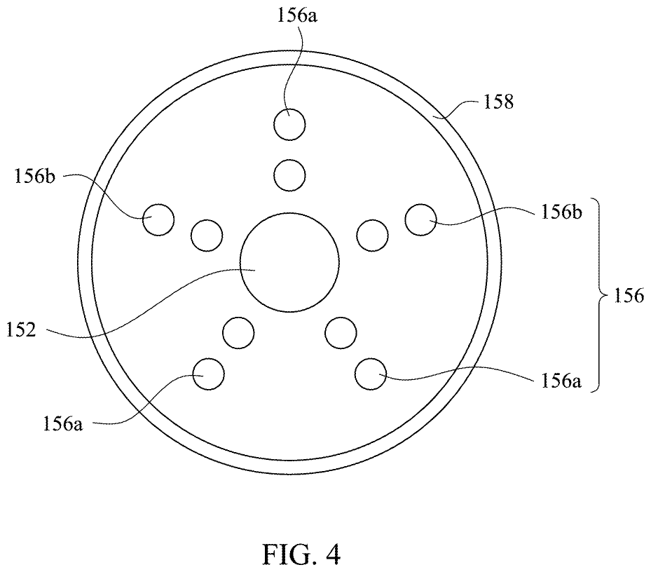

Referring to FIG. 2 and FIG. 4 simultaneously, FIG. 4 is a schematic front view of an image-taking lens, a light source module, and a cover of a mobile device for detecting an oral pathology in accordance with one embodiment of the present invention. In some examples, the mobile device 100 for detecting an oral pathology may further include a detection unit 150. The detection unit 150 may be used to inspecting if the teeth of the examinate have dental plaque. For example, the detection unit 150 may be used to check if the examinate get a periodontal disease caused by the dental plaque.

The detection unit 150 may mainly include an image-taking lens 152, an image-taking module 154, and a light source module 156. The image-taking lens 152 is disposed on one side of the casing 110, for example, both the image-taking lens 152 and the probing unit 120 are connected to the front end of the casing 110, as shown in FIG. 1. The image-taking lens 152 may be used to get various oral images in a mouth cavity of the examinate. When the examinate has dental plaque, these oral images include dental plaque images. In some exemplary examples, the image-taking lens 152 may be used to take at least five oral images in different areas in a mouth cavity of each examinate to completely determine if the teeth in the various areas of the mouth cavity have dental plaque.

Referring to FIG. 1 and FIG. 2 simultaneously, the image-taking module 154 is disposed within the casing 110 to form an embedded module. The image-taking module 154 is in signal connection with the image-taking lens 152. For example, the image-taking module 154 may achieve signal connection with the image-taking lens 152 by a wired method using an electric wire, or may be in signal connection with the image-taking lens 152 by a wireless transmission method. Thus, the image-taking lens 152 can transmit the taken oral images to the image-taking module 154. After obtaining the oral images, the image-taking module 154 can perform an image processing on the oral images. For example, the image-taking module 154 may increase image contrast, perform Gaussian equalization on the images, and/or mark and count dental plaque. In some exemplary examples, the image-taking module 154 may be a CMOS image-taking module.

Referring to FIG. 4 continuously, the light source module 156 is adjacent to the image-taking lens 152, and may provide the image-taking lens 152 with light while the image-taking lens 152 takes the oral images. For example, the light source module 156 may surround the image-taking lens to provide the image-taking lens 152 with more uniform light from various positions surrounding the image-taking lens 152. In some examples, the light source module 156 includes various first light-emitting diodes 156a and various second light-emitting diodes 156b, in which the first light-emitting diodes 156a and the second light-emitting diodes 156b alternate with each other and surround the image-taking lens 152. In addition, the first light-emitting diodes 156a and the second light-emitting diodes 156b have different emission wavelengths, i.e. the wavelengths emitted by the first light-emitting diodes 156a and the second light-emitting diodes 156b are different. In some exemplary examples, the first light-emitting diodes 156a may be ultraviolet light-emitting diodes, and the second light-emitting diodes 156b may be white light-emitting diodes. For example, the emission wavelength of the first light-emitting diodes 156a may be 405 nm. In other examples, the light source module 156 may include at least three light sources having different emission wavelengths according to requirements.

The detection unit 150 may use a fluorescence principle of quantitative light-induced fluorescence (QLF) to enhance bacteria images in the mouth cavity. For example, ultraviolet light emitted by the first light-emitting diodes 156a may be used to excite the bacteria to make the bacteria generate autofluorescence, such that the dental plaque image in the taken oral cavity image is clearer. In addition, the oral cavity images taken by the image-taking lens 152 can be color images by using the second light-emitting diodes 156b that emit white light.

Referring to FIG. 2 and FIG. 4 continuously, in some examples, the mobile device 100 for detecting an oral pathology may optionally include a cover 158. The cover 158 is disposed around the image-taking lens 152 and the light source module 156 to surround the image-taking lens 152 and the light source module 156, and places ahead the image-taking lens 152 and the light source module 156. The cover 158 can collect the light emitted by the light source module 156 around the image-taking lens 152 to enhance a capturing effect of the oral images of the image-taking lens 152.

In some examples, the image-taking module 154 is in signal connection with the processor 130. The image-taking module 154 is in signal connection with the image-taking lens 152. For example, the image-taking module 154 may be in signal connection with the processor 130 by a wired method using an electric wire, or by a wireless transmission method. Thus, the image-taking module 154 can transmit the received oral images, or the oral images which are image processed or marked with dental plaque by the image-taking module 154 to the processor 130. The processor 130 can determine the detection result of a dental plaque state of each tooth in the mouth cavity according to the received oral images. The processor 130 may further fill the detection result of the dental plaque states of the teeth out a periodontal digital medical record form via the healthcare information system and the information and communications technology system.

In these examples, by additionally installing the detection unit 150 for inspecting dental plaque in the mobile device 100 for detecting an oral pathology, the detection unit 150 can be firstly used to check if teeth of an examinate get a periodontal disease caused by the dental plaque, and then the probing unit 120 can be used to detect if the teeth of the examinate have gingivitis. Therefore, the mobile device 100 for detecting an oral pathology can do more comprehensive oral detection to the examinate.

Referring to FIG. 1 continuously, in some examples, the mobile device 100 for detecting an oral pathology may further optionally include a display device 160. The display device 160 is disposed on another side of the casing 110, in which the display device 160 and the image-taking lens 152 are located on different sides of the casing 110. For example, the display device 160 and the image-taking lens 152 are located on two opposite sides of the casing 110, in which the image-taking lens 152 is located on the front side of the casing 110, and the display device 160 is located on the rear side of the casing 110. The display device 160 may be in signal connection with the processor 130 by a wired transmission method or a wireless transmission method similarly. Thus, the processor 130 can transmit the states of the gum portion 142 of each tooth 140 probed by the probing unit 120 and the images of the mouth cavity detected by the detection unit 150 to the display device 160. The display device 160 can display the states of the gum portion 142 of each tooth 140 and the images of the mouth cavity after receiving, such that it is beneficial for a detector to know the states of the gum portion 142 of each tooth 140 of the examinate and to view the oral image to know a dental plaque condition of each tooth 140 of the examinate. In some exemplary examples, the display device 160 may be a touch screen for operation benefit of the detector.

Referring to FIG. 1 continuously, in some examples, the mobile device 100 for detecting an oral pathology further includes a handle 170 optionally. The handle 170 is connected to the casing 110. The handle 170 may be connected to a side of the casing 110 which is convenient for the detector to take, such that it is beneficial for the detector to hold the mobile device 100 for detecting an oral pathology to detect. For example, the handle 170 may be connected to a lower side of the casing 110.

According to the aforementioned embodiments, one advantage of the present invention is that a probing unit of a mobile device for detecting an oral pathology of the present invention recognizes an inflamed tissue feature of a gum by using a diffuse reflection principle, such that it needs not to be inserted into a gum space while detecting a gum state, thereby significantly easing discomfort of an examinate.

According to the aforementioned embodiments, another advantage of the present invention is that a mobile device for detecting an oral pathology of the present invention may include a detection unit for inspecting dental plaque, such that the mobile device can firstly check if the teeth get a periodontal disease caused by the dental plaque, and then detect gingivitis. Therefore, the mobile device for detecting an oral pathology can do more comprehensive oral detection to an examinate.

According to the aforementioned embodiments, still another advantage of the present invention is that a processor of a mobile device for detecting an oral pathology of the present invention can be connected to a healthcare information system and an information and communications technology system of a hospital, and can fill the relative probe and detection data out periodontal digital medical record forms. Therefore, time required for filling out the form in detection is greatly reduced, and it is beneficial to perfect a treatment solution of an examinate after detecting.

Although the present invention has been described in considerable detail with reference to certain embodiments thereof, the foregoing embodiments of the present invention are illustrative of the present invention rather than limiting of the present invention. It will be apparent to those having ordinary skill in the art that various modifications and variations can be made to the present invention without departing from the scope or spirit of the invention. Therefore, the spirit and scope of the appended claims should not be limited to the description of the embodiments contained herein.

What is claimed is:

1. A mobile device for detecting an oral pathology, comprising:
a casing;
a probing unit connected to the casing and comprising at least one fiber bundle set and at least one contact part, wherein each of the at least one contact part is configured to contact a gum portion of a tooth of an examinate, and each of the at least one fiber bundle set comprises:
a light source fiber bundle having a light-exiting end, wherein the light-exiting end is disposed within the at least one contact part, and the light source fiber bundle is configured to project a probing light onto the gum portion; and
a light-receiving fiber bundle adjacent to the light source fiber bundle and having a light-receiving end, wherein the light-receiving end is disposed within the at least one contact part, and the light-receiving fiber bundle is configured to receive diffuse reflection lights which are generated after the probing light is diffuse reflected by the gum portion; and
a processor disposed within the casing and in signal connection with the light-receiving fiber bundle, wherein the processor is configured to receive the diffuse reflection lights, to build an optical spectrum by using the diffuse reflection lights, and to determine a state of the gum portion according to the optical spectrum.

2. The mobile device for detecting an oral pathology of claim 1, wherein the at least one contact part is a sheath structure, and the at least one contact part is configured to cap the tooth of the examinate and cover the gum portion.

3. The mobile device for detecting an oral pathology of claim 2, wherein a quantity of the at least one fiber bundle set is 6, and a quantity of the at least one contact part is 1, the fiber bundle sets are equally divided into two groups which are respectively disposed in two opposite sides of the contact part, and the two sides of the contact part respectively correspond to a buccal side and a lingua side of the tooth.

4. The mobile device for detecting an oral pathology of claim 1, wherein the light source fiber bundle is an infrared light source fiber bundle, and the probing light is an infrared light.

5. The mobile device for detecting an oral pathology of claim 1, wherein the processor stores a gingival diffuse reflection spectrum data base, and the processor matches the optical spectrum using the gingival diffuse reflection spectrum data base to determine the state of the gum portion.

6. The mobile device for detecting an oral pathology of claim 1, wherein the processor is configured to connect to a healthcare information system and an information and communications technology system, and the processor is further configured to fill the state of the gum portion out a periodontal digital medical record form via the healthcare information system and the information and communications technology system.

7. The mobile device for detecting an oral pathology of claim 1, further comprising a detection unit, wherein the detection unit comprises:
an image-taking lens disposed on one side of the casing and configured to get a plurality of oral images of the examinate;
an image-taking module disposed within the casing and in signal connection with the image-taking lens to obtain the oral images; and
a light source module adjacent to the image-taking lens and configured to provide the image-taking lens with light.

8. The mobile device for detecting an oral pathology of claim 7, wherein the light source module comprises a plurality of first light-emitting diodes and a plurality of second light-emitting diodes, the first light-emitting diodes and the second light-emitting diodes alternate with each other and surround the image-taking lens, and the first light-emitting diodes and the second light-emitting diodes have different emission wavelengths.

9. The mobile device for detecting an oral pathology of claim 8, wherein the first light-emitting diodes are a plurality of ultraviolet light-emitting diodes, and the second light-emitting diodes are a plurality of white light-emitting diodes.

10. The mobile device for detecting an oral pathology of claim 7, further comprising:
a display device disposed on another side of the casing and in signal connection with the processor, wherein the display device is configured to display the oral images and the state of the gum portion;
a handle connected to the casing; and
a cover disposed around the image-taking lens and the light source module.
